(19) United States
(12) Patent Application Publication
(10) Pub. No.: US 2026/0207130 A1
(43) Pub. Date: Jul. 23, 2026

(54) IMPROVED METHOD AND DEVICE FOR ASSESSING DRUG COMPOUND NEEDS OF PATIENTS, AND ASSOCIATED DELIVERY DEVICE

(71) Applicants: UNIVERSITE DE LILLE, LILLE (FR); CENTRE HOSPITALIER UNIVERSITAIRE DE LILLE, LILLE (FR); INSERM (INSTITUT NATIONAL DE LA SANTÉ ET DE LA RECHERCHE MÉDICALE, PARIS (FR)

(72) Inventors: Julien DE JONCKHEERE, LOME (FR); Régis LOGIER, MARQUETTE LEZ LILLE (FR); Idir IBARISSENE, Lille (FR); Michel DELECROIX, WATTIGNIES (FR); Mathieu JEANNE, Lille (FR)

(73) Assignees: UNIVERSITE DE LILLE, LILLE (FR); CENTRE HOSPITALIER UNIVERSITAIRE DE LILLE, LILLE (FR); INSERM (INSTITUT NATIONAL DE LA SANTÉ ET DE LA RECHERCHE MÉDICALE, PARIS (FR)

(21) Appl. No.: 19/142,259

(22) PCT Filed: Dec. 18, 2023
(86) PCT No.: PCT/EP2023/086345

§ 371 Date: Jun. 23, 2025

(30) Foreign Application Priority Data
Dec. 21, 2022 (FR) 2214151

(51) Int. Cl. A61B 5/00; A61B 5/021; A61B 5/024

(52) U.S. Cl. CPC A61B 5/4824; A61B 5/021; A61B 5/02405; A61B 5/4839

(57) ABSTRACT

The invention relates to a method (100) for evaluating the needs of a drug compound of a patient, comprising a step of determining (150), based on MC and ML indices derived from an ANI index, and their slopes PC and PL, and physiological data relating to the patient, the need for a drug compound required by the patient, comprising a sub-step (151) of determining a bolus, comprising fuzzy logic for determining fuzzy logic variables C_{MC} and C_{PC}, the bolus being equal to a first predetermined amount of said compound when the sum of the first and second fuzzy logic variable is strictly greater than 1, the bolus being equal to zero otherwise. The invention also relates to a device for assessing a patient's needs for a drug compound, as well as a device (300) for delivering a drug compound, comprising such an assessment device (200).

### RELATED APPLICATIONS

This application is a national stage of PCT application No. PCT/EP2023/086345, filed on Dec. 18, 2023; and the PCT application claimed the benefit of priority of French Application No. 2214151, filed on Dec. 21, 2022. The contents of the foregoing documents are incorporated herein by reference in entirety.

### TECHNICAL FIELD

The present disclosure relates to the technical field of managing the pain of a patient.

More specifically, the present disclosure relates to an improved method for assessing the needs of a patient for a medicinal compound. The invention also relates to an improved device for assessing the needs of a patient for a medicinal compound, as well as a device for delivering medicinal compounds comprising in particular such an assessment device. The drug compound is in particular an analgesic and/or hypnotic compound.

In particular, the invention finds applications in the management of a patient's pain during a surgery on a patient under general or local anaesthesia. According to other possibilities, the invention may find applications more broadly for the management of pain of conscious or unconscious patients in hospitals, in particular in intensive care or palliative care, in care facilities, or at home.

### BACKGROUND

When managing a patient's pain, it is necessary to assess the level of pain accurately and in real time, so that a medical response can be provided that is adapted to the patient's needs.

For example, the medical response may consist of injecting an analgesic, antalgic or pain-relieving compound into the patient.

However, the amount of compound injected into the patient should be proportionate and customised for the patient according to his/her condition and physiological characteristics.

In order to assess the pain level of a patient, the determination of an ANI (the acronym for “Analgesia Nociception Index”), determined according to the variability of the patient's heart rate, is known. The determination of such an ANI index is known in the art, in particular by means of the devices and methods known from patent documents FR 2821460, FR 2840187, FR 2864388 and EP 1804655 filed by the Applicant.

In order to assess the need for analgesic and/or hypnotic compounds in real time and taking into account the change over time in the patient's pain level, as well as the type of pain experienced by the patient, the Applicant has developed a device and method for assessing the needs for analgesic and/or hypnotic compounds that takes into account averages of the ANI index over short and long periods, as well as the slope of the ANI index over these periods.

In particular, these techniques have been the subject of patents FR 3006879 and FR 3006880 by the Applicant.

The aforementioned techniques provide for the determination of an amount of analgesic and/or hypnotic compound to be administered to the patient at a point in time. Such an amount of compound, significant compared with the amount of compound normally administered, and intended to be administered over a short period of time, is called a “bolus” in medical terminology.

However, there has been a need for a more stable and repeatable technique for assessing drug compound requirements, particularly with regard to determining a bolus to be administered to the patient.

The need has also arisen to determine more finely, i.e. more precisely and closer to the patient's instantaneous compound requirements, the amount of compound a patient requires. This is because some known techniques underestimate or overestimate the need for compound, resulting in an increased risk of hypotension or hypertension for the patient.

### SUMMARY

The present invention aims to overcome all or some of the aforementioned drawbacks of the existing technology.

To this end, the invention relates to a method for assessing the needs of a patient for a medicinal compound, intended to be implemented by an assessment device comprising means for measuring the cardiac variability of the patient between 0.15 Hz and 4 Hz, a data processing and recording unit, and means for recording physiological data relating to the patient, the method comprising the following steps:
a. a step of measuring the patient's cardiac variability between 0.15 Hz and 4 Hz;
b. a step of first determining, by the data processing and recording unit, from the measured cardiac variability data, an ANI index relating to the pain level of the patient;
c. a step of second determination by the data processing and recording unit, from the ANI index, of a MC index, representative of the average of the ANI index over a so-called short period, and an ML index, representative of the average of the ANI index over a so-called long period, as well as the PC and PL slopes respectively of said MC and ML indices, the MC and ML indices corresponding to the pain level of a patient and the PC and PL indices as it evolves, over a short period and a long period respectively;
d. a step of recording physiological data relating to the patient, by the means for recording physiological data relating to the patient;
e. a step of third determination by the data processing and recording unit, according to the MC and ML indices, their PC and PL slopes, and physiological data relating to the patient, the need for a medicinal compound required by the patient, said third determination step comprising a sub-step of determining an amount of said compound to be administered to the patient, called a bolus, comprising:
f. determining a first fuzzy logic variable C_{MC}, lying between 0 and 1, from a membership function F_{MC} of the MC index,
g. determining a second fuzzy logic variable C_{PC}, lying between 0 and 1, from a membership function F_{PC} of the index PC,
h. determining that the bolus is equal to a first predetermined amount of said compound when the sum of the first fuzzy logic variable C_{MC} and the second fuzzy logic variable C_{PC} is strictly greater than 1, and that the bolus is equal to zero otherwise,
Said third determination step further comprising a sub-step of determining a second amount of said compound to be administered, less than said first predetermined amount, determined according to the indices MC and ML, their slopes PC and PL, and physiological data relating to the patient, this sub-step being executed by the method following the sub-step of determining a bolus when the bolus is equal to zero.

The method according to the invention is based on a fuzzy logic step.

The determination of a bolus by means of a fuzzy logic substep makes it possible to greatly improve the stability and the repeatability of the assessment of requirement for compound, compared with the techniques known in the art.

The sub-step of determining an amount of the compound to be administered at isolated times to the patient, i.e. a sub-step of determining a bolus, makes it possible to evaluate whether or not a bolus can be administered to the patient.

If a bolus can be administered to the patient with respect to the MC and PC threshold values, the bolus value corresponds to a predetermined amount of compound. The predetermined amount of compound may be fixed, or be defined upstream of the implementation of the method, for example according to data relating to the patient.

If a bolus cannot be administered to the patient with respect to the MC and PC threshold values, the bolus value is zero.

The calculation of the average of the ANI index can be performed on a sliding window. In particular in such a case, the repeatability of the method according to the invention is even more important compared with the known technique.

These aspects make it possible to provide a process that is efficient and safe, and that meets the requirements for placing such a process implemented by a medical device on the market.

It is specified here that the method can operate in a loop, in real time or periodically, i.e. said steps of the method are repeated in succession so as to determine in real time or periodically the need for medicinal compounds of the patient.

In the context of clinical trials of the method according to the invention, a decrease in the amount of analgesic and/or hypnotic compound to be administered to patients, and better haemodynamic stability, i.e. patients are less prone to hypotension and hypertension, was observed compared with known methods.

According to a second aspect, the invention also relates to a device for assessing the needs of a patient for a medicinal compound, comprising means for measuring the cardiac variability of the patient between 0.15 Hz and 4 Hz, a data processing and recording unit, and means for recording physiological data relating to the patient, the processing and recording unit comprising a processor and a computer memory storing instructions configuring the processor to:
i. receive data on the patient's cardiac variability between 0.15 Hz and 4 Hz;
j. determine, from the received cardiac variability data, an ANI index relative to the level of pain of the patient;
k. determine, from the ANI index, a MC index representative of the average of the ANI index over a so-called short period, and an ML index, representative of the average of the ANI index over a so-called long period, as well as the PC and PL slopes respectively of said MC and ML indices, the MC and ML indices corresponding to the pain level of a patient and the PC and PL indices as it evolves, over a short period and a long period respectively;
l. receive physiological data relating to the patient;
m. determine, based on the MC and ML indices, their PC and PL slopes, and physiological data relating to the patient, the need for a drug compound to be administered to said patient, this step comprising a sub-step of determining a first amount of said compound to be administered as a single dose to the patient, called a bolus, comprising:
n. determine a first fuzzy logic variable C_{MC}, between 0 and 1, from a membership function F_{MC} of the MC index,
o. determine a second fuzzy logic variable C_{PC}, comprised between 0 and 1, from a membership function F_{PC} of the index PC,
p. determine that the bolus is equal to a first predetermined amount when the sum of the first fuzzy logic variable C_{MC} and the second fuzzy logic variable C_{PC} is strictly greater than 1, and that the bolus is equal to zero otherwise
q. determine a second amount of said compound to be administered, less than said first predetermined amount, according to the MC and ML indices, their PC and PL slopes, and physiological data relating to the patient, following the determination of a bolus, when the bolus is equal to zero.

Such a device has the technical advantages and effects of the method according to the invention described above.

The device for assessing the needs of the patient for a drug compound can in particular be in the form of a computer having inputs to which a cardiac variability sensor and at least one other sensor for physiological data of the patient are connected.

The assessment device may comprise a device for displaying data, in particular of an amount of drug compound to be administered.

It is specified that the method, and the device, may also be a method, and a device, for assessing the needs for a drug compound, or drugs, and/or paramedical care of a patient. Preferably, the drug compound is an analgesic and/or hypnotic compound.

Other particularly advantageous and convenient features of the method according to the invention, and of the assessment device according to the invention, are described below.

According to one embodiment, in the assessment method and device according to the invention, the membership function F_{MC} of the MC index is:
r. equal to 1 when the MC index is less than or equal to a low threshold value S_{MCLow},
s. equal to 0 when the MC index is greater than or equal to a high threshold value S_{MCHigh},
t. and has a monotonic decreasing transition between 1 and 0 when the MC index is between the low S_{MCLow} and high threshold values S_{MCHigh}, and wherein the membership function F_{PC} of the PC index is: membership function is:
u. equal to 1 when the PC index is less than or equal to a low threshold value S_{PCLow},
v. S_{PCHigh} equal to 0 when the PC index is greater than or equal to a high threshold value,
w. and has a monotonic decreasing transition between 1 and 0, when the PC index is between the low S_{PCLow} and high threshold values S_{PCHigh}.

The choice of this type of membership function made it possible to observe a good performance of the method, which is particularly stable and repeatable.

The type of monotonic decreasing transition may be adapted according to the type of compound intended to be administered, and/or according to the clinical situation of the patient. Likewise, the high and low thresholds can be adapted according to these same criteria.

According to one embodiment, in the method and the assessment device according to the invention, the function of membership F_{MC} of the MC index is defined according to the expression:

$${F}_{MC} = \{ \begin{matrix}1 , & if MC \leq {S}_{MCLow} \\ \frac{MC - {S}_{MCHigh}}{{S}_{MCLow} - {S}_{MCHigh}} , & if {S}_{MCLow} < MC < {S}_{MCHigh} \\ 0 , & if MC \geq {S}_{MCHigh}\end{matrix}$$

and the PC F_{PC} index membership function is defined according to the expression:

$${F}_{PC} = \{ \begin{matrix}1 , & if PC \leq {S}_{PCLow} \\ \frac{PC - {S}_{PCHigh}}{{S}_{PCLow} - {S}_{PCHigh}} , & if {S}_{PCLow} < PC < {S}_{PCHigh} \\ 0 , & if PC \geq {S}_{PCHigh}\end{matrix} .$$

In general, a decreasing linear transition allows good performance of the method.

Such a transition is particularly suitable during surgery during which the patient is under general anaesthesia.

According to one embodiment, in the assessment method and device according to the invention:
x. the threshold value S_{MCLow} is between 45 and 55, and preferably equal to 50,
y. the threshold value S_{MCHigh} is between 58 and 68, and preferably equal to 63,
z. the threshold value S_{PCLow} is between −40 and −30 and preferably equal to −35,
aa. the threshold value S_{PCHigh} is between −30 and −20, and preferably equal to −25.

Threshold ranges, as well as preferential thresholds, are particularly suitable during surgery where the patient is under general anaesthesia.

These values are particularly suitable when the drug compound is an analgesic and/or hypnotic compound which is remifentanil.

According to one embodiment, in the method according to the invention, the step of recording physiological data relating to the patient is a step of recording at least one systolic blood pressure (SBP) of the patient, by the physiological-data recording means that are configured to measure and record at least one systolic blood pressure (SBP), the sub-step of determining a bolus being performed by the method when the patient's systolic blood pressure (SBP) is greater than or equal to at least one of a first systolic blood pressure threshold value S_{SBP1} and a second systolic blood pressure threshold value S_{SBP2}.

In the assessment device according to the invention, receiving physiological data relating to the patient comprises receiving at least one systolic blood pressure (SBP) data of the patient, for example acquired by the physiological-data recording means which are configured to measure and record at least one systolic blood pressure (SBP), the determination of a bolus being performed when the systolic blood pressure (SBP) of the patient is greater than or equal to at least one of a first systolic blood pressure threshold value S_{SBP1} and a second systolic blood pressure threshold value S_{SBP2}.

According to one embodiment, in the assessment method and device according to the invention, the first systolic blood pressure threshold value S_{SBP1} corresponds to a predetermined mean value of systolic blood pressure, and the second systolic blood pressure threshold value S_{SBP2} corresponds to a previously measured and recorded systolic blood pressure of the patient.

In particular, in the method according to the invention, the second systolic blood pressure threshold value S_{SBP2} corresponds to a systolic blood pressure of the patient measured and recorded prior to the execution of said method.

By such setting of the thresholds, it is possible to adapt the proportional regulation according to a generally accepted mean systolic blood pressure, and a systolic blood pressure of the patient prior to the execution of the process.

According to one embodiment, in the method according to the invention, the systolic blood pressure of the patient measured and recorded previously corresponds to a systolic blood I pressure of the patient measured and recorded during a previous execution of said method.

In the assessment device according to the invention, the previously measured and recorded systolic blood pressure of the patient corresponds to a systolic blood pressure of the patient obtained during a previous reception of the physiological data of the patient by the assessment device.

When the method according to the invention is executed in a loop, or when the assessment device assesses the needs in a loop, the proportional regulation can take into account the systolic blood pressure measured and recorded during a previous iteration of the method or operation of the assessment device, thereby making it possible to follow the change in the systolic blood pressure.

According to one embodiment, the method comprises a step of proportional regulation of an amount of drug compound to be administered, performed by the method upstream of the step of third determination, when the patient's systolic blood pressure (SBP) is strictly lower than both said first threshold value S_{SBP1} and said second systolic blood S_{SBP2} pressure threshold value, said regulation step comprising determining an amount of drug compound to be administered corresponding to an amount of previously administered drug compound, from which an amount of drug compound directly proportional to a change in the measured and recorded value of the patient's systolic blood pressure (SBP) is subtracted, with respect to a value of the patient's systolic blood pressure measured and recorded prior to the execution of said method.

In the assessment device according to the invention, the instructions comprise the proportional regulation of an amount of medicinal compound to be administered, carried out upstream of the determination of the compound requirement, when the patient's systolic blood pressure (SBP) is strictly lower than both said first threshold value S_{SBP1} and said second systolic blood pressure threshold value S_{SBP2}, said regulation comprising determining an amount of drug compound to be administered corresponding to an amount of previously administered drug compound, from which is subtracted an amount of drug compound directly proportional to a change in the measured and recorded value of the patient's systolic blood pressure (SBP), compared with a value of the patient's systolic blood pressure received during a previous assessment by the assessment device.

By such regulation proportional to the decrease in systolic blood pressure, the amount of compound to be administered can be reduced, which decreases the risk of hypotension for the patient. In addition, the total amount of compound to be administered may be reduced, and more adapted to the patient's condition.

Thus, when the patient's systolic blood pressure drops too much, the amount of drug compound is reduced.

Here, it may be an amount of drug compound determined prior to any use of the method or assessment by the assessment device, or an amount of drug compound determined during the step of third determination of said method, during a previous execution of the method, or prior determination of the need for compound by the assessment device.

When the method is executed in a loop, or the assessment device evaluates in a loop, it is understood that the determination of the compound requirement, and therefore the determination of a bolus, is carried out only when the regulation, executed a sufficient number of times, has made it possible to verify the condition according to which the systolic blood pressure is no longer lower than one of the two aforementioned systolic blood pressure thresholds.

This makes it possible to secure the method or use of the assessment device, ensuring that no excessive amount of compound, and in particular no bolus, risks being administered to a patient in a hypotension situation.

According to a third aspect, the invention also relates to a device for delivering a drug compound, comprising a device for assessing the needs of a patient for a drug compound according to a second aspect of the invention, associated with control means and a perfusion system, preferably electrical.

The delivery device may in particular comprise control means configured to regulate a flow rate of a drug compound according to the amount of the determined drug compound, via an action on the perfusion system.

### BRIEF DESCRIPTION OF THE DRAWINGS

Other particular advantages, aims and features of the present invention will become apparent from the following non-limiting description of at least one particular embodiment of the devices and methods objects of the present invention, with reference to the appended drawings:

FIG. 1 shows schematically a delivery device comprising an assessment device according to the invention;

FIG. 2 shows schematically an assessment device according to the invention;

FIG. 3 is a flow diagram of the assessment method according to the invention;

FIG. 4 is a flow diagram of the assessment method according to the invention, comprising an optional step of proportional regulation;

FIG. 5 shows the graph of the F_MC membership function of the MC index and the graph of the F_PC membership function of the PC index; and

FIG. 6 is a flow diagram of the assessment method according to one embodiment.

### DETAILED DESCRIPTION

The present description is given as a non-limiting example, each feature of one embodiment could be advantageously combined with any other feature of any other embodiment.

As of now, it should be noted that the figures are not plotted to scale.

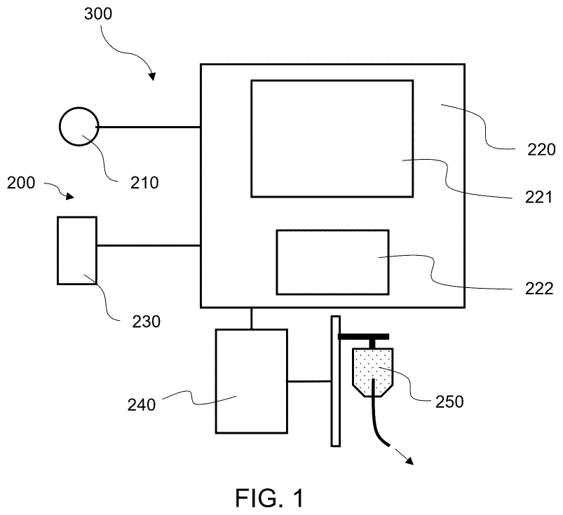

FIG. 1 shows schematically a compound-delivery device 300.

The delivery device 300 comprises a device 200 for assessing drug compound needs, control means 240, and a perfusion system 250.

Preferably, the perfusion system 250 is electrical, and is actuated by the control means 240, connected by a data link to the assessment device 200. The perfusion system is suitable for dispensing a drug compound into the blood circuit of a patient requiring administration of the drug compound, for example via a catheter of the perfusion system 250.

The drug compound is in particular an analgesic and/or hypnotic compound.

In particular, the analgesic and/or hypnotic compound is an analgesic compound, for example an opioid compound, in particular morphine. For example, the compound is remifentanil. According to other examples, the compound is a non-morphinic compound such as ketamine, or a compound of the alpha-2-agonist class, such as clonidine.

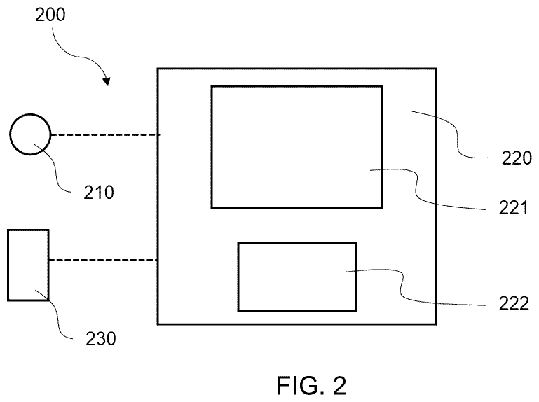

FIG. 2 shows schematically an assessment device 200 alone, i.e. not associated in particular with control means 240 and a perfusion system 250. The assessment device 200 can in fact operate alone without means of administering the compound, determining the needs for analgesic and/or hypnotic and/or curarizing compound and transmitting this information to another device, or to medical personnel for example.

The assessment device 200 may in particular comprise means 210 for measuring the cardiac variability of the patient between 0.15 Hz and 4 Hz, a data processing and recording unit 220 and means 230 for recording physiological data relating to the patient.

For example, the data processing and recording unit 220 comprises a microprocessor, or processor, 221 and a computer memory 222, adapted to store instructions configuring the processor 221 for operation thereof. For example, the computer memory 222 is a non-volatile storage computer memory, which can be rewritable, being for example of the “EPROM” type.

The means 230 for recording physiological data relating to the patient may be physiological data sensors, in particular commercially available sensors. These sensors may be configured to record physiological, or vital, data of the patient, and transmit them via a data link to the data processing and recording unit 220. For example, the recording means 230 comprise at least one blood pressure sensor, in particular systolic pressure, also called a blood pressure monitor.

The means 210 for measuring the cardiac variability of the patient between 0.15 Hz and 4 Hz may comprise various means known to those skilled in the art, such as an electrocardiograph (ECG) and/or an oximeter, through which the cardiac variability of the patient between 0.15 Hz and 4 Hz can be determined.

The assessment device 200 may also comprise other peripherals, such as information display means, for example a computer screen.

The assessment device 200 makes it possible to assess, based on physiological data of the patient, to assess a need for an analgesic and/or hypnotic compound of a patient, hereinafter referred to as “compound”, in particular when the patient is placed under anaesthesia or sedation.

The need for compound may in particular be the determination of an amount of compound required by the patient. The assessment device 200 can make it possible to communicate such an amount of compound to medical personnel, for example via a computer screen, or to communicate the amount of compound to the control means 240 for administration by the perfusion system 250.

Thus the assessment device 200 makes it possible to make a recommendation on the requirements of a patient for a compound, and the appropriate amount of compound can then be administered by the medical staff, or automatically by the perfusion system, at the decision of the medical staff.

The need for compounds is assessed by the assessment device 200 in particular from an ANI index, which is an index relating to the level of pain experienced by the patient. Thus the need for compound can be determined according to a perceived level of pain, and thus assess the optimal amount of compound needed by the patient to relieve pain, without under- or overdosing the amount of compound.

For this purpose, the assessment device 200 is configured to implement a method 100 for assessing the needs of a patient for an analgesic and/or hypnotic compound.

More specifically, the computer memory 222 of the assessment device 200 stores instructions configuring the processor 221 to implement the steps of the method 100.

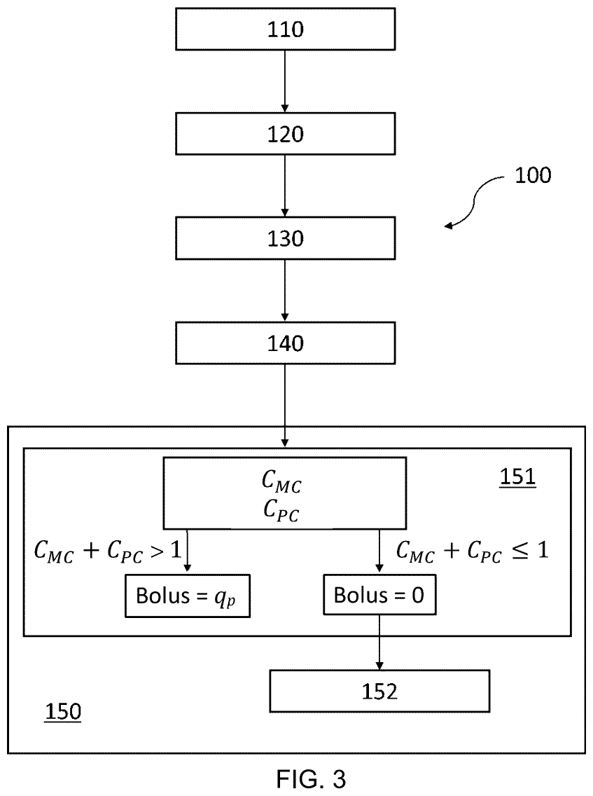

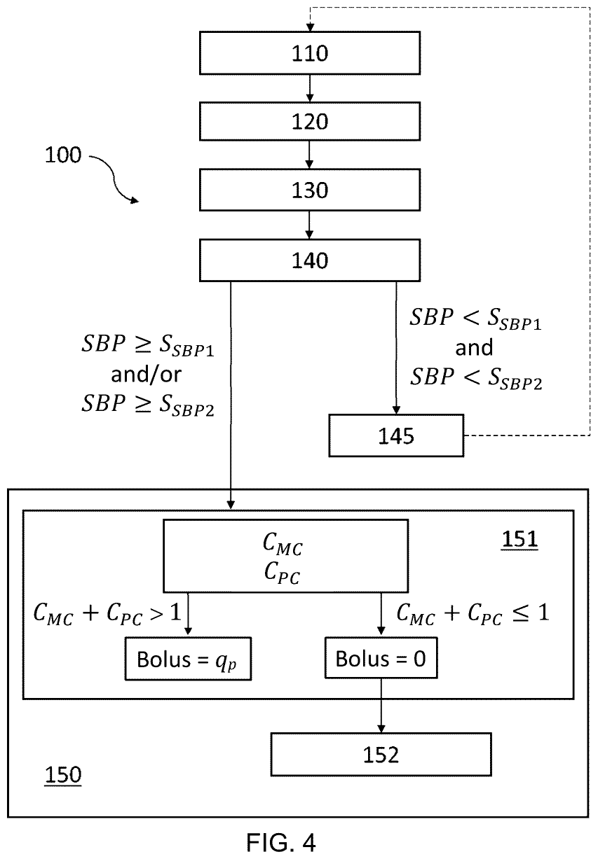

The method 100, intended to be implemented by the assessment device 200, will now be described, with reference to FIGS. 3 and 4 in particular.

The method 100 comprises a step 110 of measuring the cardiac variability of the patient between 0.15 Hz and 4 Hz.

The method 100 then comprises a step of first determining 120, by the data processing and recording unit 220, from the cardiac variability data measured during the step 110, an ANI index relative to the pain level of the patient.

For example, the ANI index may be calculated according to the method described in application WO 2006/032739. The first determination step 120 can thus comprise the steps of such a method in order to determine the ANI index.

The method 100 further comprises a step 130 of secondly determining by the data processing and recording unit 220 a sub-index, or index, MC and a sub-index, or index, ML.

The MC and ML indices are determined from the ANI index, the MC index being representative of the average of the ANI index over a so-called short period, and the ML index being representative of the average of the ANI index over a so-called long period.

The MC and ML indices therefore correspond to the level of pain of a patient, respectively over a so-called short period and a so-called long period, of duration greater than the short period.

Furthermore, in the second determination step 130, the slopes PC and PL respectively of said indices MC and ML, corresponding to the change in the pain level of a patient, respectively over the short period and the long period, are determined.

In other words, the MC and ML indices are calculated from the various values of the ANI over a given period of time, and make it possible to limit the impact of the instantaneous variability of the ANI measurement due to the calculation method and also to derive trends in the change in the patient's pain.

More specifically, the calculation of the MC and ML indices is preferably carried out from an average of the values of the ANI indices over two different periods, namely a short period and a long period.

Advantageously, the short period corresponds to a sliding window of 45 to 90 seconds and preferably of the order of 60 seconds, while the long period corresponds to a sliding window of 90 to 240 seconds and preferably of the order of 180 seconds.

Advantageously, the period taken into account for the calculation of ML is substantially of the order of 1.5 to 3 times the period used for the calculation of MC.

The PC slope is calculated in degrees from two separate MC values of a given duration according to the following formula:

$$PC = ( \frac{180}{π} ) arc tan ( \frac{M C ( t 2 ) - M C ( t 1 )}{t 2 - t 1} ) .$$

Advantageously, the time interval between t1 and t2 is of the order of 15 to 45 seconds and preferably equal to 30 seconds.

The PL slope is calculated in degrees in a similar way to PC according to the following formula:

$$PL = ( \frac{180}{π} ) arc tan ( \frac{M L ( t 2 ) - M L ( t 1 )}{t 2 - t 1} ) .$$

Again, the time interval separating t1 and t2 is of the order of 15 to 45 seconds and preferably equal to 30 seconds.

In addition, the method 100 comprises a step 140 of recording physiological data relating to the patient, by means 230 for recording physiological data relating to the patient.

As presented earlier, the recording means 230 allow for example the measurement of the heart rate (HR) and the systolic blood pressure (SBP).

These parameters are not used directly in the calculation of the optimal amount of compounds required by the patient, but are used in such a way as to constitute safeguards, as the assessment device does not perform an assessment when certain thresholds corresponding to these parameters are exceeded.

An example of a safety step will be described later in this description.

In other embodiments, other physiological parameters may be used in substitution for or in addition to heart rate (HR) and systolic blood pressure (SBP).

It is also possible, although this mode would have the disadvantage of degraded safety, to use only one physiological parameter.

It is also possible to record by the recording means 230 other types of data relating to the patient such as weight, height, age or gender of the patient. For this purpose, the recording means 230 may advantageously comprise a keyboard or touch surface, not shown in the appended figures.

The method 100 also comprises a step of third determination 150 by the data processing and recording unit 220 of the need for a medicinal compound, here for example an analgesic and/or hypnotic compound, required by the patient

The need for analgesic and/or hypnotic compound required by the patient is determined based on the MC and ML indices, their PC and PL slopes, and physiological data relating to the patient.

More specifically, during the step of third determination 150, it is first determined whether the patient requires the administration of a first amount qₚ of said compound, to be administered to the patient in a single dose. Such a relatively large amount of compound, and intended to be administered over a short time, is called a “bolus”. The patient requires a compound bolus when the pain is acute, i.e. when the ANI index is close to 0.

For this purpose, the third determination step 150 comprises a sub-step 151 of determining a first amount of the compound to be administered in a single dose to the patient, i.e. a sub-step 151 of determining a bolus.

The sub-step 151 of determining a bolus comprises determining a first fuzzy logic variable C_{MC}, between 0 and 1, from a membership function F_{MC} of the MC index.

The sub-step 151 of determining a bolus also comprises determining a second fuzzy logic variable C_{PC}, between 0 and 1, from a membership function F_{PC} of the index PC.

The sub-step 151 of determining a bolus then comprises determining that the bolus is equal to a first predetermined amount of qₚ said compound when the sum of the first fuzzy logic variable C_{MC} and the second fuzzy logic variable C_{PC} is strictly greater than 1, and that the bolus is equal to zero otherwise.

In other words, the patient requires a bolus when the sum of the first fuzzy logic variable C_{MC} and the second fuzzy logic variable C_{PC} is strictly greater than 1, and does not require a bolus when the sum of the first fuzzy logic variable C_{MC} and the second fuzzy logic variable C_{PC} is less than 1.

Sub-step 151 of determining a bolus is thus a step calling on fuzzy logic to determine the necessity of administering or not administering a bolus.

The MC index membership function F_{MC} and PC index membership function F_{PC} are here functions bounded between 0 and 1. Thus the image of an index value MC or PC by the respective membership function is a value C_{PC} or C_{MC} between 0 and 1, i.e. a “fuzzy” Boolean value.

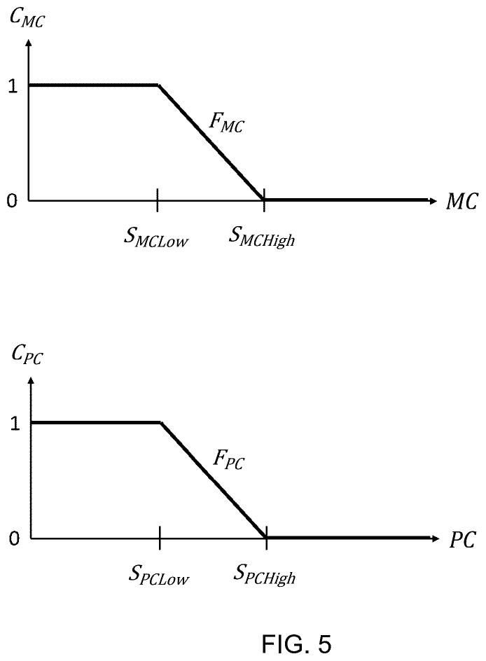

FIG. 5 shows the graphs of the MC index membership function F_{MC} and PC index membership function F_{PC}.

For example, the MC index membership function F_{MC} is:
S_{MCLow}—Equal to 1 when the MC index is less than or equal to a low threshold value;
S_{MCHigh}—Equal to 0 when the MC index is greater than or equal to a high threshold value;
And has a monotonic decreasing transition between 1 and 0, when the MC index is between the low S_{MCLow} and high S_{MCHigh} threshold values,
Similarly, for example, the PC index membership function F_{PC} is:
Equal to 1 when the PC index is less than or equal to a low threshold value S_{PCLow};
Equal to 0 when the PC index is greater than or equal to a high threshold value S_{PCHigh};
And has a monotonic decreasing transition between 1 and 0, when the PC index is between the low S_{PCLow} and high S_{PCHigh} threshold values.

Examples of a monotonic decreasing transition are a linear, hyperbolic, in particular sigmoid, exponential or even Gaussian transition.

A linear decreasing transition is particularly preferred.

For example, the MC index membership function F_{MC} is defined by the expression

$${F}_{MC} = \{ \begin{matrix}1 , & if MC \leq {S}_{MCLow} \\ \frac{MC - {S}_{MCHigh}}{{S}_{MCLow} - {S}_{MCHigh}} , & if {S}_{MCLow} < MC < {S}_{MCHigh} : \\ 0 , & if MC \geq {S}_{MCHigh}\end{matrix} .$$

For example, the PC index membership function F_{PC} is defined according to the expression

$${F}_{PC} = \{ \begin{matrix}1 , & if PC \leq {S}_{PCLow} \\ \frac{PC - {S}_{PCHigh}}{{S}_{PCLow} - {S}_{PCHigh}} , & if {S}_{PCLow} < PC < {S}_{PCHigh} \\ 0 , & if PC \geq {S}_{PCHigh}\end{matrix} .$$

The high and low transition thresholds S_{MCLow}, S_{MCHigh}, S_{PCLow} and S_{PCHigh} can be freely adapted according to the conditions of use of the method 100, in particular according to the patient profile (in particular their age, weight, gender, etc.), and/or the type of compound intended to be administered to the patient, and/or the patient's clinical condition, i.e. according to their medical care needs. Similarly, the type of transition can be freely adapted according to these same criteria.

For example, the threshold values S_{MCLow}, S_{MCHigh}, S_{PCLow} and S_{PCHigh} can be set as follows:
The threshold value S_{MCLow} is between 45 and 55, and preferably equal to 50.
The threshold value S_{MCHigh} is between 58 and 68, and preferably equal to 63.
The threshold value S_{PCLow} is between −40 and −30 and preferably equal to −35.
The threshold value S_{PCHigh} is between −30 and −20, and preferably equal to −25.

Such ranges of values, and preferential values, in particular associated with a linear transition, are particularly suitable during surgery under general anaesthesia, when remifentanil as a compound is to be administered to the patient. Indeed, repeatable and stable operation of the method could be observed in this context.

The sub-step 151 of determining a bolus, based on fuzzy logic, makes it possible to obtain the determination of a particularly reliable and repeatable bolus, even when the MC index has a high variability, in particular when it is determined on a sliding window. This is because fuzzy logic makes it possible to dispense with a bolus determination based on fixed thresholds, which is much more sensitive to variations in the MC index.

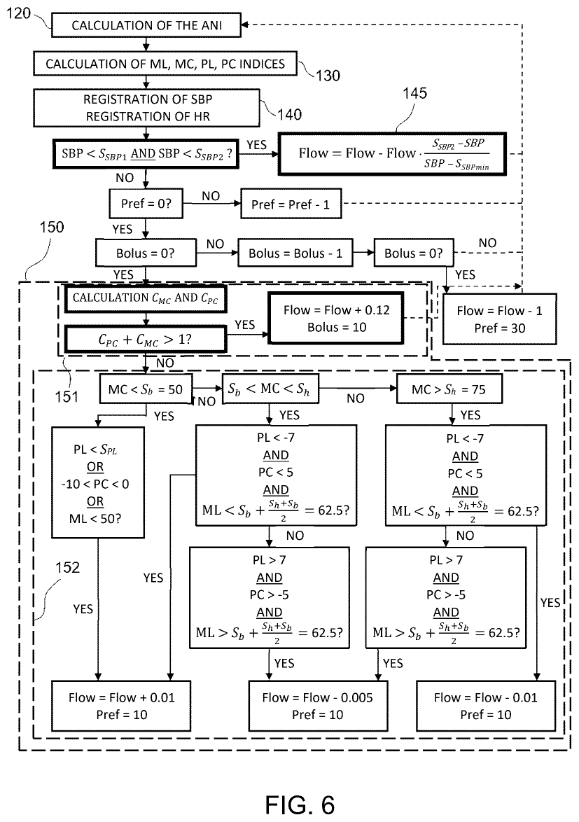

Concretely, with reference to FIG. 6, which shows a more detailed example of implementation of the method 100 in the form of a flow diagram, when a bolus is determined, and when the compound used is remifentanil with a concentration of 25 μg/ml, a flow-rate variable is for example increased according to the weight of the patient by 0.12 μg/kg/min for 10 seconds.

In this example, a “Bolus” variable is set to 10, this variable being decremented during a next execution of the method 100, which can operate in a continuous or periodic loop. As long as the variable “Bolus” is not zero, no new step of determining an amount of compound is performed by the method 100.

If, at the end of the sub-step 151 of determining a bolus, the sum of the first fuzzy logic variable C_{MC} and the second fuzzy logic variable C_{PC} is less than 1, i.e. the bolus is zero, the method 100 executes a sub-step 152 of determining a second amount of the compound to be administered, less than said first predetermined amount qₚ.

The second amount of the compound is determined based on the MC and ML indices, their PC and PL slopes, and physiological data relating to the patient.

For example, the sub-step 152 of determining a second amount of the compound comprises a series of successive comparisons of the indices MC, ML, PC and PL with predetermined thresholds.

In particular, as can be seen in FIG. 6 at sub-step 152, the MC index is first compared successively with low S_{b} and high Sₕ thresholds, in order to determine whether the mean of the ANI index over a short period is low, moderate, or high, i.e. if the pain is severe, moderate, or low.

Based on this first determination, based on the trend of the mean pain level over a long period, and the change in the main pain level over the short and long periods, the method 100 determines the second amount of compound. The second amount of compound may be greater or less than a second amount of compound determined previously, in particular determined during a previous execution of the method 100. It is also possible that the second amount of compound remains unchanged relative to a second amount of compound determined previously.

Once the sub-step 152 is executed, and the second amount of compound is determined, the method 100 can be executed again from the beginning. It is possible to delay a new execution of the method, for example by setting a value of the variable “Pref”, here at 10 in the example of FIG. 6, this variable being decremented during each execution of the method, before the execution of the third determination step 150.

The specific implementation of the sub-step 152, as well as the specific operation of the decrementation loops of the variables “Bolus” and “Pref” of the algorithm illustrated in FIG. 6, are described in the patent documents FR 3006879 and FR 3006880 of the Applicant and to which a person skilled in the art may refer for the implementation of the method 100.

As discussed earlier in the present description, the method 100 may comprise a safety step, aimed at preventing any determination of amount of compound to be administered when certain thresholds of physiological data of the patient are exceeded.

For example, with reference to FIGS. 4 and 6, the method 100 comprises an optional step of comparing the systolic blood pressure (SBP) with two systolic blood pressure thresholds.

For this purpose, the step 140 of recording physiological data relating to the patient is then a step of recording at least one systolic blood pressure (SPB) of the patient, by the means 230 for recording physiological data, which are then configured to measure and record at least one systolic blood pressure (SPB).

The optional step of comparing systolic blood pressure (SBP) with two systolic blood pressure thresholds comprises comparing the systolic blood pressure (SBP) with:
A first systolic blood pressure threshold S_{SBP1}, which may be a predetermined mean value of systolic blood pressure, for example a systolic blood pressure of 120 mmHg which may be considered as a minimum systolic blood pressure threshold of a healthy person, and with:
A second systolic blood pressure threshold S_{SBP2}, which may be a systolic blood pressure (SBP) value of the patient, measured and recorded prior to the execution of the method 100. For example, the patient's systolic blood pressure (SBP) may have been measured and recorded during a previous execution of the method 100.

When the patient's systolic blood pressure (SBP) is greater than or equal to at least one of the first systolic blood pressure threshold value S_{SBP1} and the second systolic blood pressure threshold value S_{SBP2}, the sub-step 151 of determining a bolus is executed by the method 100.

This is because, when the patient's systolic blood pressure (SBP) is not lower than the first threshold, and does not decrease compared with a previous systolic blood pressure, it is possible to consider that the patient's risk of hypotension is low, and that a quantity of compound to be administered can be determined without danger to the patient's health.

When the patient's systolic blood pressure (SBP) is strictly lower than both the first threshold value S_{SBP1} and the second systolic blood pressure threshold value S_{SBP2}, a step 145 of proportional regulation of an amount of analgesic and/or hypnotic compound to be administered is performed by the method upstream of the step of third determination 150. The step of third determination 150, and thus in particular the determination of a bolus, is then not executed by the method 100 during the same iteration of the method 100.

The step 145 of proportional regulation comprises determining an amount of analgesic and/or hypnotic compound to be administered, this amount corresponding to an amount of previously administered analgesic and/or hypnotic compound, from which is subtracted an amount of analgesic and/or hypnotic compound directly proportional to a change in the measured and recorded value of the patient's systolic blood pressure (SBP), relative to a value of the patient's systolic blood pressure measured and recorded prior to the execution of the method 100.

Specifically, with reference to the algorithm illustrated in FIG. 6, a compound flow rate setpoint variable “Flow” may be updated directly proportionally to the variation of the measured and recorded value of the patient's systolic blood pressure (SBP), relative to a value of the patient's systolic blood pressure measured and recorded prior to the execution of the method 100.

For example, a new flow setpoint can be defined as follows:

$$Flow = Flow - Flow \cdot \frac{{S}_{SBP 2} - SBP}{SBP - {S}_{S BPm i n}} ,$$

where S_{SBPmin} is a predetermined cut-off value for systolic blood pressure, for example corresponding to 80 mmHg.

In other words, the “Flow Rate” variable is overwritten and updated so as to reduce the compound flow rate setpoint proportionally to the change in the patient's systolic blood pressure (SBP).

The proportional-regulation step 145 is performed in a loop until the patient's systolic blood pressure is no longer below one of the two threshold values S_{SBP1} and S_{SBP2}, i.e. the risk of hypotension for the patient can be considered low. This is a safety measure, avoiding assessing excessively high compound requirements for a patient in a situation of hypotension, or pressure drop.

For example, the interval between two iterations, i.e. between two measurements of the patient's systolic blood pressure, is between 2 and 5 minutes.

In addition, the proportional-regulation step 145 allows for a better adaptation of the delivery of the compound according to the needs of the patient, and also makes it possible to decrease the total amount of compound to be administered to the patient, which is both clinically and economically advantageous.

Although the optional step of comparing the systolic blood pressure (SBP) with two systolic blood pressure thresholds, followed where appropriate by a step 145 of proportional regulation, is particularly advantageous as presented above, it can also be envisaged providing a more conventional safety step as described in the patent documents FR 3006879 and FR 3006880 of the Applicant. The safety step described therein provides for a stoppage of compound flow when the patient is hypotensive or bradycardic.

According to one embodiment, the method 100, and/or the assessment device 200, and/or the delivery device 300, may be intended for the assessment of a need for paramedical care of a patient, alternatively or complementarily to the assessment of the need for a drug compound.

In other words, in addition to or as an alternative to determining an amount of compound required by the patient, it is possible to assess the paramedical care needs of a patient in a similar manner, i.e. based on a fuzzy logic based on an ANI index and its sub-indices. For example, determining a need for paramedical care comprises determining that paramedical care is required by the patient, and may for example comprise determining the duration and/or intensity of paramedical care.

“Paramedical care” means all care that can be provided to a patient, with the exception of medical and surgical treatments, including, but not limited to, paediatric care, physiotherapy, neonatology, neurophysiology, or more broadly, care based on changing the environment to improve patient wellbeing.

More generally, it should be recalled that the invention is not limited to the described and illustrated examples.

1-10. (canceled)

11. A method for assessing needs of a patient for a medicinal compound implemented by an assessment device, wherein the assessment device comprises a unit for measuring a patient's cardiac variability between 0.15 Hz and 4 Hz, a data processing and recording unit, and a unit for recording physiological data relating to the patient, the method comprising the following steps:
measuring the cardiac variability of the patient between 0.15 Hz and 4 Hz;
determining by the data processing and recording unit, based on measured cardiac variability data, an Analgesia Nociception Index (ANI index) relating to a pain level of the patient;
determining by the data processing and recording unit, based on the ANI index, an MC index representative of a mean of the ANI index over a short period, an ML index representative of an average of the ANI index over a long period, and a PC slope and a PL slope respectively of said MC and ML indices, the MC and ML indices corresponding to the pain level of the patient, and the PC and PL indices corresponding to a change in the pain level over the short period and a change in the pain level over the long period respectively;
recording physiological data relating to the patient by the unit for recording physiological data relating to the patient; and
determining, by the data processing and recording unit, based on the MC and ML indices, the slopes PC and PL, and the physiological data relating to the patient, a need for a medicinal compound required by the patient, wherein the determining of the need for the medicinal compound required by the patient comprises:
determining a first amount of said medicinal compound to be administered to the patient in a single dose as a bolus, comprising:
determining a first fuzzy logic variable C_{MC}, between 0 and 1, forming a membership function F_{MC} of the MC index,
determining a second fuzzy logic variable C_{PC}, between 0 and 1, forming a membership function F_{PC} of the index PC, and
determining that the bolus is equal to a first predetermined amount of said compound when a sum of the first fuzzy logic variable C_{MC} and the second fuzzy logic variable C_{PC} is strictly greater than 1, and that the bolus is equal to zero otherwise, wherein the determining of the need for the medicinal compound required by the patient further comprises:
determining a second amount of said medicinal compound to be administered, less than said first predetermined amount, determined according to the indices MC and ML, the slopes PC and PL, and the physiological data relating to the patient, wherein this step of determining the second amount of said medicinal compound is executed the step of determining the first amount of said medicinal compound in response to that the bolus is zero.

12. The method according to claim 11, wherein the membership function F_{MC} of the MC index is:
equal to 1 when the MC index is less than or equal to a low threshold value S_{MCLow},
equal to 0 when the MC index is greater than or equal to a high threshold value S_{MCHigh}, and
and the membership function has a monotonic decreasing transition between 1 and 0, when the MC index is between the low S_{MCLow} and high S_{MCHigh} threshold values, and wherein the PC index membership function F_{PC} is:
equal to 1 when the PC index is less than or equal to a low threshold value S_{PCLow},
S_{PCHigh} equal to 0 when the PC index is greater than or equal to a high threshold value, and
the membership function has a monotonic decreasing transition between 1 and 0, when the PC index is between the low S_{PCLow} and high S_{PCHigh} threshold values.

13. The method according to claim 12, wherein the membership function F_{MC} of the MC index is defined according to an expression:
$${F}_{MC} = \{ \begin{matrix}1 , & if MC \leq {S}_{MCLow} \\ \frac{MC - {S}_{MCHigh}}{{S}_{MCLow} - {S}_{MCHigh}} , & if {S}_{MCLow} < MC < {S}_{MCHigh} \\ 0 , & if MC \geq {S}_{MCHigh}\end{matrix}$$
and the PC index membership function F_{PC} is defined according to an expression:
$${F}_{PC} = \{ \begin{matrix}1 , & if PC \leq {S}_{PCLow} \\ \frac{PC - {S}_{PCHigh}}{{S}_{PCLow} - {S}_{PCHigh}} , & if {S}_{PCLow} < PC < {S}_{PCHigh} \\ 0 , & if PC \geq {S}_{PCHigh}\end{matrix} .$$

14. The method according to claim 12, wherein
the threshold value S_{MCLow} is between 45 and 55,
the threshold value S_{MCHigh} is between 58 and 68,
the threshold value S_{PCLow} is between −40 and −30, and
the threshold value S_{PCHigh} is between −30 and −20.

15. The method according to claim 14, wherein
the threshold value S_{MCLow} is equal to 50,
the threshold value S_{MCHigh} is equal to 63,
the threshold value S_{PCLow} is equal to −35, and
the threshold value S_{PCHigh} is equal to −25.

16. The method according to claim 13, wherein
the threshold value S_{MCLow} is between 45 and 55,
the threshold value S_{MCHigh} is between 58 and 68,
the threshold value S_{PCLow} is between −40 and −30, and
the threshold value S_{PCHigh} is between −30 and −20.

17. The method according to claim 16, wherein
the threshold value S_{MCLow} is equal to 50,
the threshold value S_{MCHigh} is equal to 63,
the threshold value S_{PCLow} is equal to −35, and
the threshold value S_{PCHigh} is equal to −25.

18. The method according to claim 11, wherein the step of recording the physiological data relating to the patient comprises recording at least one systolic blood pressure (SBP) of the patient, by the unit of recording physiological data, and the unit of recording physiological data is configured to measure and record the at least one systolic blood pressure (SBP), the determining of the bolus is performed when the patient's systolic blood pressure (SBP) is greater than or equal to at least one of a first systolic blood pressure threshold value S_{SBP1} or a second systolic blood pressure threshold value S_{SBP2}.

19. The method according to claim 18, wherein the first systolic blood pressure threshold value S_{SBP1} corresponds to a predetermined mean value of systolic blood pressure, and the second systolic blood pressure threshold value S_{SBP2} corresponds to a systolic blood pressure of the patient.

20. The method according to claim 19, wherein a previously measured and recorded systolic blood pressure of the patient corresponds to a previously measured and recorded systolic blood pressure of the patient during a previous execution of said method.

21. The method according to claim 15, wherein the method further comprises a step of proportional regulation of an amount of the medicinal compound to be administered, performed prior to the determining of the need of the medicinal compound when the patient's systolic blood pressure (SBP) is strictly lower than both said first threshold value S_{SBP1} and said second systolic blood pressure threshold value S_{SBP2},
the step of proportional regulation comprises: determining an amount of the medicinal compound to be administered corresponding to an amount of the medicinal compound previously administered, from which an amount of the medicinal compound directly proportional to a change in a measured and recorded value of the patient's systolic blood pressure (SBP) is subtracted, with respect to a value of the patient's systolic blood pressure measured and recorded prior to the execution of said method.

22. The method according to claim 16, wherein the method further comprises a step of proportional regulation of an amount of the medicinal compound to be administered, performed prior to the determining of the need of the medicinal compound when the patient's systolic blood pressure (SBP) is strictly lower than both said first threshold value S_{SBP1} and said second systolic blood pressure threshold value S_{SBP2},
the step of proportional regulation comprises: determining an amount of the medicinal compound to be administered corresponding to an amount of the medicinal compound previously administered, from which an amount of the medicinal compound directly proportional to a change in a measured and recorded value of the patient's systolic blood pressure (SBP) is subtracted, with respect to a value of the patient's systolic blood pressure measured and recorded prior to the execution of said method.

23. The method according to claim 17, wherein the method further comprises a step of proportional regulation of an amount of the medicinal compound to be administered, performed prior to the determining of the need of the medicinal compound when the patient's systolic blood pressure (SBP) is strictly lower than both said first threshold value S_{SBP1} and said second systolic blood pressure threshold value S_{SBP2},
the step of proportional regulation comprises: determining an amount of the medicinal compound to be administered corresponding to an amount of the medicinal compound previously administered, from which an amount of the medicinal compound directly proportional to a change in the measured and recorded value of the patient's systolic blood pressure (SBP) is subtracted, with respect to a value of the patient's systolic blood pressure measured and recorded prior to the execution of said method.

24. A device for assessing needs of a patient for a medicinal compound, comprising:
a unit for measuring a patient's cardiac variability between 0.15 Hz and 4 Hz;
a data processing and recording unit; and
a unit for recording physiological data relating to the patient, wherein the processing and recording unit comprises a processor and a memory storing instructions configuring the processor to perform:
receiving data on the patient's cardiac variability between 0.15 Hz and 4 Hz,
determining, based the received cardiac variability data, an ANI index relative to a level of pain of the patient,
determining by the data processing and recording unit, based on the ANI index, an MC index representative of a mean of the ANI index over a short period, an ML index representative of an average of the ANI index over a long period, and a PC slope and a PL slope respectively of said MC and ML indices, the MC and ML indices corresponding to the pain level of the patient, and the PC and PL indices corresponding to a change in the pain level over the short period and a change in the pain level over the long period respectively,
receiving physiological data relating to the patient,
determining, based on the MC and ML indices, based on the MC and ML indices, the slopes PC and PL, and the physiological data relating to the patient, a need for a medicinal compound required by the patient, wherein the determining of the need for the medicinal compound required by the patient comprises:
determining a first amount of said medicinal compound to be administered to the patient in a single dose as a bolus, comprising:
determining a first fuzzy logic variable C_{MC}, between 0 and 1, forming a membership function F_{MC} of the MC index,
determining a second fuzzy logic variable C_{PC}, between 0 and 1, forming a membership function F_{PC} of the index PC, and
determining that the bolus is equal to a first predetermined amount of said compound when a sum of the first fuzzy logic variable C_{MC} and the second fuzzy logic variable C_{PC} is strictly greater than 1, and that the bolus is equal to zero otherwise, wherein the determining of the need for the medicinal compound required by the patient further comprises:
determining a second amount of said medicinal compound to be administered, less than said first predetermined amount, determined according to the indices MC and ML, the slopes PC and PL, and the physiological data relating to the patient, wherein this step of determining the second amount of said medicinal compound is executed the step of determining the first amount of said medicinal compound in response to that the bolus is zero.

25. A device for delivering a medicinal compound, comprising:
an assessment device;
a control unit; and
an infusion system, wherein the assessment device is configured to assess needs of a patient for a medicinal compound, comprising:
a unit for measuring a patient's cardiac variability between 0.15 Hz and 4 Hz;
a data processing and recording unit; and
a unit for recording physiological data relating to the patient, wherein the processing and recording unit comprises a processor and a memory storing instructions configuring the processor to perform:
receiving data on the patient's cardiac variability between 0.15 Hz and 4 Hz,
determining, based the received cardiac variability data, an ANI index relative to a level of pain of the patient,
determining by the data processing and recording unit, based on the ANI index, an MC index representative of a mean of the ANI index over a short period, an ML index representative of an average of the ANI index over a long period, and a PC slope and a PL slope respectively of said MC and ML indices, the MC and ML indices corresponding to the pain level of the patient, and the PC and PL indices corresponding to a change in the pain level over the short period and a change in the pain level over the long period respectively,
receiving physiological data relating to the patient,
determining, based on the MC and ML indices, based on the MC and ML indices, the slopes PC and PL, and the physiological data relating to the patient, a need for a medicinal compound required by the patient, wherein the determining of the need for the medicinal compound required by the patient comprises:
determining a first amount of said medicinal compound to be administered to the patient in a single dose as a bolus, comprising:
determining a first fuzzy logic variable C_{MC}, between 0 and 1, forming a membership function F_{MC} of the MC index,
determining a second fuzzy logic variable C_{PC}, between 0 and 1, forming a membership function F_{PC} of the index PC, and
determining that the bolus is equal to a first predetermined amount of said compound when a sum of the first fuzzy logic variable C_{MC} and the second fuzzy logic variable C_{PC} is strictly greater than 1, and that the bolus is equal to zero otherwise, wherein the determining of the need for the medicinal compound required by the patient further comprises:
determining a second amount of said medicinal compound to be administered, less than said first predetermined amount, determined according to the indices MC and ML, the slopes PC and PL, and the physiological data relating to the patient, wherein this step of determining the second amount of said medicinal compound is executed the step of determining the first amount of said medicinal compound in response to that the bolus is zero.

26. The device according to claim 25, wherein the infusion system comprises an electrical infusion system.
